(19) United States
(12) Patent Application Publication
(10) Pub. No.: US 2004/0253720 A1
(43) Pub. Date: Dec. 16, 2004

(54) Use of the cd24 marker gene for the selection of keratinocyte stem cells transformed by exogenous sequences

(76) Inventors: Thierry Magnaldo, Paris (FR); Alain Sarasin, Thiais (FR)

Correspondence Address: YOUNG & THOMPSON, 745 SOUTH 23RD STREET, 2ND FLOOR, ARLINGTON, VA, 22202, US

(21) Appl. No.: 10/482,641

(22) PCT Filed: Jul. 4, 2002
(86) PCT No.: PCT/FR02/02347

(30) Foreign Application Priority Data
Jul. 6, 2001 (FR) 01/08984

(51) Int. Cl. C12N 5/08; C12N 15/85

(52) U.S. Cl. 435/371; 435/455

(57) ABSTRACT

A subject of the present invention is the use of the CD24 marker gene of mammals, such as the human CD24 gene, for the transformation of stem keratinocytes in the implementation of processes for preparing homogenous batches of epidermal stem cells transformed in order to contain an exogenous nucleotide sequence of interest, as well as said stem keratinocytes thus transformed, the epidermal tissue reconstituted starting with these keratinocytes, and their uses.

A subject of the present invention is processes for preparing homogenous batches of stem keratinocytes transformed with an exogenous sequence of interest, as well as said thus transformed stem keratinocytes, the epidermal tissue reconstituted from these keratinocytes, and their uses.

The approaches to gene transfer in keratinocytes which are currently proposed are confronted with problems which result:

either from selection of the transduced cells in the presence of a lethal pharmacological agent (type G418); these techniques still rely on a gene resistant to a lethal pharmacological agent (neomycin, hygromycin, blasticidine, zeocine, etc.),

or from the non-selection of the transduced cells

Two major drawbacks exist in using a gene resistant to a lethal pharmacological agent, and this problem seems to be all the more pronounced in the case of epidermal keratinocytes:

a-) the product of a gene of exogenous origin, namely of the above-mentioned resistance gene, expressed by the transduced cells can be recognised as nonself. Thus this produces the problem of immune response if the transduced cells are intended for grafting onto an immunocompetent individual, i.e. the strong probability of a rejection of the graft in the long term.

b-) depletion during the selection of the growth potential of the transduced cells, which in itself limits the chances of engraftment, of maintenance and of long term survival of any tissue graft produced from these genetically modified cells.

The transduction of the keratinocytes in the absence of selection allows the problems set out above to be bypassed: immune response and depletion of the growth potential during the selection.

However the absence of selection brings problems:

of the homogeneity of the population of transduced cells,

of the absence of selection on the criterion of the capacity for proliferation of the transduced cells.

These two problems are incompatible with perspectives of cell and tissue therapy which rely on keratinocytes, in particular on keratinocytes from patients suffering from Xeroderma pigmentosum (XP). A keratinocyte population transduced in the absence of selection will necessarily contain a low % of non-transduced cells, i.e. cells in which sensitivity to UV remains. The persistence of these non-transduced cells in the culture, in a reconstructed epidermis and optionally in a graft, will be translated by the non-correction of the phenotype with predisposition to cutaneous carcinomas and therefore the reimplantation of potentially tumorous cells.

On the other hand in the absence of selection, no growth advantage is given to the transduced cells relative to the non-transduced cells. In fact a single division is necessary and sufficient for retroviral infection. The long term survival of a graft produced from transduced cells in the absence of selection could therefore be inferior to that of a graft carried out from cells selected on the basis of their ability to proliferate as is proposed in the present invention.

For several years the Inventors have demonstrated that the UV-sensitive phenotype of the XP fibroblasts in culture of patients suffering from xeroderma pigmentosum can be reversed by transfer of the gene with suitable complementation (Carreau et al., 1994; Quilliet et al., 1996; Zheng et al., 1998). More precisely, these studies have demonstrated that the reintroduction of an XP gene restores the capacity of the cells to repair their DNA to the point of recovering their normal survival to UV. Nevertheless these studies have

a-) been carried out on dermal fibroblasts, cells which are only exceptionally (<1%) at the origin of cutaneous cancers, including in XP patients;

b-) used retroviral vectors equipped with the gene resistant to neomycin and which therefore cannot be used for perspectives of reversion of epidermal XP keratinocytes.

An object of the invention is to homogeneously select human epidermal cells (keratinocytes) in culture into which an exogenous gene has been introduced. These genetically modified cells can either be used for experimental purposes, or grafted in the donor subject after reconstitution of the epidermal tissue in vitro. The major advantage of the invention relative to the system of selection which is already known is found in the natural character of the selection marker and therefore the ability to reimplant the transduced cells without the patient rejecting an unknown exogenous substance.

The invention follows from the Inventors demonstrating that in the epidermis only the post-mitotic keratinocytes naturally express the CD24 marker at their surface, whereas the stem keratinocytes do not express this CD24 marker.

It should be noted that CD24 is naturally expressed in the epidermis. This is what makes the difference between the use of CD24 versus selection markers which are used in a standard fashion (genes resistant to a pharmacological agent, in particular to an antibiotic such as G418 the resistance of which is carried by the bacterial resistance gene neo), since, in contrast to CD24, these are not naturally expressed in the epidermal keratinocytes or in the epidermis and therefore subject to immune reaction, or even to rejection of the graft. More precisely, as mentioned above, CD24 is naturally expressed in the postmitotic suprabasal layers (which have lost the ability to divide) of the human epidermis, but is not expressed at a detectable level in the cells of the basal layer of this tissue. The principle of the invention is based on this specificity of expression of CD24 inside the tissue and the recognition of the differential proliferation properties of the basal and suprabasal compartments of the epidermal cells. In fact, the basal cells of the epidermis are the only ones capable of multiplying in vivo and are at the origin of the permanent renewal of the tissue. Moreover, these cells can be cultivated in vitro. The epidermal basal cells' capacity to multiply in culture makes them susceptible to retroviruses and in particular to the retroviruses derived from Moloney's murine leukemogenic virus (MoMLV) produced by packaging cells which produce infectious particles with amphotropic covers.

Thus the invention aims to bypass the above-mentioned limitations to the extent that it is based on the use of a cell surface marker, CD24 which is:

a-) naturally-occurring in the epidermis,

b-) the expression of which does not interfere with the ability of the epidermal keratinocytes to proliferate,

c-) advantageously under control of the transcriptional promoter contained in the LTR 5′ of the proviral DNA, i.e. the expression of which will disappear approximately 4 weeks after grafting of an epidermis reconstructed from the transduced cells (Choate et al., 1997).

The principle of the invention is based on a novel process for selecting keratinocytes expressing the exogenous gene of interest through the coexpression of the latter, with a surface marker, CD24. The two genes (gene of interest or exogenous gene, and CD24 ) are present in the same construction of proviral DNA. The transcriptional control of each of these genes is ensured by two independent promoters (for example LTR 5′ for the gene CD24, and an internal “physiological” promoter such as for example the promoter of the human keratin K5 gene for the transcriptional control of the gene of interest).

The principle of selection is therefore applicable to the keratinocytes in cultures within the scope of the present invention. In a keratinocyte culture derived from the human epidermis, two cellular populations will be found schematically. One population capable of multiplying and not expressing CD24. This population can therefore be infected by the viral amphotropic particles; a population not multiplying itself, involved in the irreversible process of differentiation and expressing CD24. During the infection of a keratinocyte culture heterogeneous in terms of proliferation and of expression of CD24, the transduction of the CD24 gene in the cells in division will lead to the ectopic expression of the corresponding protein (CD24). At the end of the retroviral infection, all of the cell population expressing CD24, either naturally (cells which are differentiated, post-mitotic or becoming post-mitotic), or exogenously (transduced cells) can be selected by cell sorting using a CD24-specific antibody. The advantage of this selection is that the cellular metabolism of the infected culture is not disturbed and therefore all its biological capacities, which will be useful for the rest of the tests, are therefore retained. Of the cells thus selected and put back into culture, only those expressing CD24 exogenously, i.e. which have the ability to proliferate, are retained in the culture dishes. Whether they are transduced or not, all the cells selected for the expression of CD24, and possessing a weak to non-existent capacity to proliferate, are eliminated during the culture after a few in vitro passages. The cells possessing a high capacity to proliferate (stem cells) and which are CD24 positive (i.e. transduced) can be used to reconstruct a genetically modified epidermal tissue.

Thus, as has been mentioned above, the subject of the present invention is to bypass the above-mentioned limitations in so far as CD24, namely the selection marker used, is,

a)—expressed naturally in the epidermis (the problem of immunological tolerance),

b)—its expression on the surface of the transduced cells allows the selection of the clonogenic keratinocytes leading to a homogenous population.

c-) the selection process allows the epidermis to be enriched with progenitor cells which favours the long term survival of any graft of epidermis from genetically corrected cells.

The advantages of the selection process of the present invention are:

immunotolerance by a host organism of a graft carried out from genetically modified cells,

the possibility of carrying out a total selection, but without depletion of the cultures by a pharmacological agent; this strategy allows the long term survival of an epidermis in culture produced from genetically modified cells to be improved,

the sorting of the transduced cells is very easy and very effective, and can be undertaken in the laboratory and without specific heavy duty equipment; if appropriate, the only specific requirement is a secondary antibody,

the construction allows exploitation of the fact of the loss of expression of the CD24 gene shortly after the grafting of an epidermis produced from the genetically modified cells. In fact, in this context, the transcriptional activity of the LTR 5′ for example, is lost 3 to 4 weeks after grafting (Choate and Khavari, 1997). This loss of activity of the LTR 5′ leads to the loss of the exogenous expression of the selection marker in the basal layer of the grafted reconstructed epidermis. This allows any risks of disturbing the physiology of the epidermal tissue which can be inherent in the expression of CD24 to be minimized.

Among the possible applications of the present invention, there may be mentioned in particular, in the field of genetics, the study of the effects of the overexpression of genes which can be involved in the homeostasis of the epidermal tissue or of their mutated version.

A possible application is the construction of human epidermis expressing a mutated copy of an XP complementation gene which leads to the hyperphotosensitivity of the tissue, or the possibility of incorporating melanocytes in the reconstructed tissue in order to evaluate the role of these cells in a photosensitive context linked to a deficiency in repair of the DNA.

A subject of the present invention is the use of the CD24 marker gene of mammals, such as the human CD24 gene, for the transformation of stem keratinocytes in the context of implementing processes for preparing homogenous batches of epidermal stem cells transformed in order to contain an exogenous nucleotide sequence of interest.

Advantageously the CD24 marker genes from mammals, and more particularly the human CD24 gene, used within the scope of the present invention, are in particular those described in the following article: Kay R, Rosten P M, Humphries R K. CD24, a signal transducer modulating B cell activation response, is a very short peptide with a glycosyl phosphatidylinositol membrane anchor. J. Immunol. (1991), 147, 1412-1416.

A subject of the invention is also a process for preparing homogenous batches of epidermal stem cells transformed in order to contain an exogenous nucleotide sequence of interest, characterized in that it comprises the following stages:

the transformation of epidermal cells in culture comprising stem keratinocytes and post-mitotic keratinocytes from the suprabasal layer of the epidermis naturally secreting the CD24 protein using an appropriate vector the genome of which comprises a recombinant sequence containing an exogenous nucleotide sequence of interest and the CD24 gene as selection marker, which leads to the obtaining of a heterogeneous batch of cells, comprising the above-mentioned stem and post-mitotic keratinocytes transformed or not transformed with the recombinant sequence of the above-mentioned vector,

selection of the cells transformed during the preceding stage and expressing at their surface the CD24 protein, using a reagent specifically recognizing this protein, such as CD24-specific antibodies, which leads to the obtaining of a heterogeneous batch of cells comprising above-mentioned stem and post-mitotic keratinocytes transformed with the recombinant sequence of the above-mentioned vector, and if appropriate, above-mentioned post-mitotic keratinocytes not transformed with said recombinant sequence,

the culture of the cells selected during the preceding stage for a period which is sufficient to ensure the elimination of the above-mentioned post-mitotic keratinocytes, transformed or not transformed with said recombinant sequence, capable of being present during the preceding stage of transformation and being incapable of dividing and therefore to be maintained in the culture medium, which leads to the obtaining of a homogenous batch of CD24⁺ stem keratinocytes containing said recombinant sequence, capable of dividing and being maintained in culture.

More particularly a subject of the invention is a process such as described above, characterized in that the exogenous nucleotide sequence of interest is chosen from:

the nucleotide sequences capable of being used in gene therapy, said sequences being in particular chosen from the DNA repair genes involved in the following syndromes: xeroderma pigmentosum, namely the XPA, XPB, XPC, XPD, XPE, XPG, XPF genes, trichotiodystrophy, namely the CSA and CSB genes, Cockayne's syndrome, namely the LG-1 gene, or the genes coding for the factors intervening in coagulation of the blood, such as the gene coding for the factor VIII in the treatment of hemophilia A, or also the gene coding for insulin in the treatment of insulin-dependent diabetes,

the nucleotide sequences capable of being used as tracer genes to monitor the development of a cell or population of transduced cells expressing this marker gene, said sequences being chosen in particular from those coding for GFP (Green Fluorescent Protein), luciferase, or for beta-galactosidase of E. coli,

the nucleotide sequences comprising a dominant genetic mutation with a phenotype effect, the effects of which on the physiology of the tissue can be studied, said sequences being chosen in particular from those mentioned above which correspond to the DNA repair genes, and being involved in one or more processes for repairing DNA,

the nucleotide sequences capable of allowing the screening of molecules of therapeutic interest for the epidermis, or of cosmetological interest, said sequences being in particular chosen from the sequences controlling the transcription of genes the expression of which is modulated after UV irradiation, in particular from the following sequences:

the promoter sequences of the c-Jun gene, of collagenase;

the sequences, optionally synthetic, comprising the ultraviolet response elements (URE) TGACAACA;

the binding sequences of the P53 protein, the expression of which is stabilized by ultraviolet radiation, in particular:

the sequences contained in the MDM2 gene,

the sequences contained in the p21/cip1/WAF gene,

the sequences, optionally synthetic, comprising one or more P53 response elements.

These sequences are advantageously placed upstream of a tracer gene (as defined above). The keratinocytes transduced and sorted according to the process described above allow the in vitro reconstruction of the epidermal tissue (three dimensionally) according to the process described hereafter. The ultraviolet irradiation of these epidermides reconstructed in vitro will bring about a modulation (negative or positive) of the expression of the tracer gene; this allows the qualitative and quantitative evaluation of the in situ effect of products such as:

cosmetic substances (in particular sunscreens),

medicaments which have a negative or positive influence on the repair of lesions induced by UV in the DNA, and therefore on the expression of the tracer gene.

Advantageously, the vector used for the transformation of cells of the epidermis in culture in the process as defined above, is chosen from retroviruses such as the retroviruses derived from the Moloney's murine leukemogenic virus, or the lentivirus-type vectors, derived from HIV (these have the benefit of allowing infection of the cells in the absence of division).

More particularly a subject of the invention is any process as defined above, characterized in that the stage of selection of the cells transformed using CD24-specific antibodies is carried out using antibodies specifically recognising the CD24 antibodies and coupled to magnetic beads, the CD24+ marked cells being separated from the non-marked cells using a magnet.

A subject of the invention is also any process as defined above, characterized in that the stage of culture of the selected cells, using a reagent specifically recognising the CD24 protein, in particular an anti-CD24 antibody, in order to eliminate the post-mitotic keratinocytes is carried out in the following way: the keratinocytes selected using the anti-CD24 antibody are inoculated according to the process of JG Rheinwald, and Green H (Serial cultivation of strains of human epidermal keratinocytes: the formation of keratinizing colonies from single cells. Cell. 1975, 6, 331-43), namely on a layer of nutritive murine fibroblasts previously lethally irradiated with gamma rays; under these conditions, the selected post-mitotic cells do not attach to the support; sub- or pre-postmitotic cells can attach themselves, but form so-called abortive colonies giving rise to post-mitotic cells which are eliminated after a culture passage. Thus, the post-mitotic cells are eliminated either immediately after inoculation (cells do not attach to the support), or after a culture passage.

The invention also relates to the stem keratinocytes of mammals, and more particularly of humans, transformed in order that their genome contains the CD24 marker gene of mammals, and more particularly the human CD24 gene.

More particularly a subject of the invention is the above-mentioned transformed stem keratinocytes, characterised in that their genome contains at least one exogenous nucleotide sequence of interest as defined above.

A subject of the invention is also homogenous batches of above-mentioned transformed stem keratinocytes, and more particularly batches of transformed stem keratinocytes the homogeneity rate of which is advantageously above approximately 99%, namely the proportion of non-transformed stem keratinocytes and/or transformed or non-transformed post-mitotic keratinocytes of which is nil or lower than approximately 1%.

The invention also relates to the transformed stem keratinocytes and the abovementioned homogenous batches of the latter, as obtained by implementing a process as defined above.

A subject of the invention is also epidermal tissue reconstituted from transformed stem keratinocytes or from homogenous batches of the latter as defined above.

The invention also relates to the grafts of reconstituted epidermal tissue as mentioned above.

More particularly a subject of the invention is the use of epidermal tissue as defined above, for the preparation of above-mentioned grafts, intended for the treatment of pathologies linked to a genetic disorder.

The techniques used for the preparation of reconstituted epidermal tissue according to the invention are described in particular in the following articles: Gallico et al., N. Engl. Med. 311, 448-451, 1984). Bell et al., 1979 Proc Natl Acad Sci USA 76, 274-1278, and Asselineau et al., 1984, Exp. Cell. Res, 159, 536-539.

A subject of the invention is also the use of transformed stem keratinocytes or of homogenous batches of the latter as defined above, or of above-mentioned reconstituted epidermal tissue, for use in:

processes for monitoring the in vitro development of a cell or population of transduced cells expressing exogenous nucleotide sequences of interest as marker genes,

processes for studying in vitro the effects on the physiology of the epidermal tissue of exogenous nucleotide sequences which are of interest corresponding to sequences comprising a dominant genetic mutation with phenotype effect,

processes for in vitro screening of molecules of therapeutic interest for the epidermis, or of cosmetological interest, capable of having an effect directly on the exogenous nucleotide sequence of interest, or indirectly on the cell physiological state induced by said exogenous nucleotide sequence of interest, the latter processes comprising a stage of placing said molecules of interest in the presence of the transformed stem keratinocytes or the above-mentioned transformed epidermal tissue.

The invention is further illustrated using the detailed description which follows of the obtaining of homogenous batches of stem keratinocytes transformed according to the invention.

1. Construction of Novel Recombinant Proviral DNA

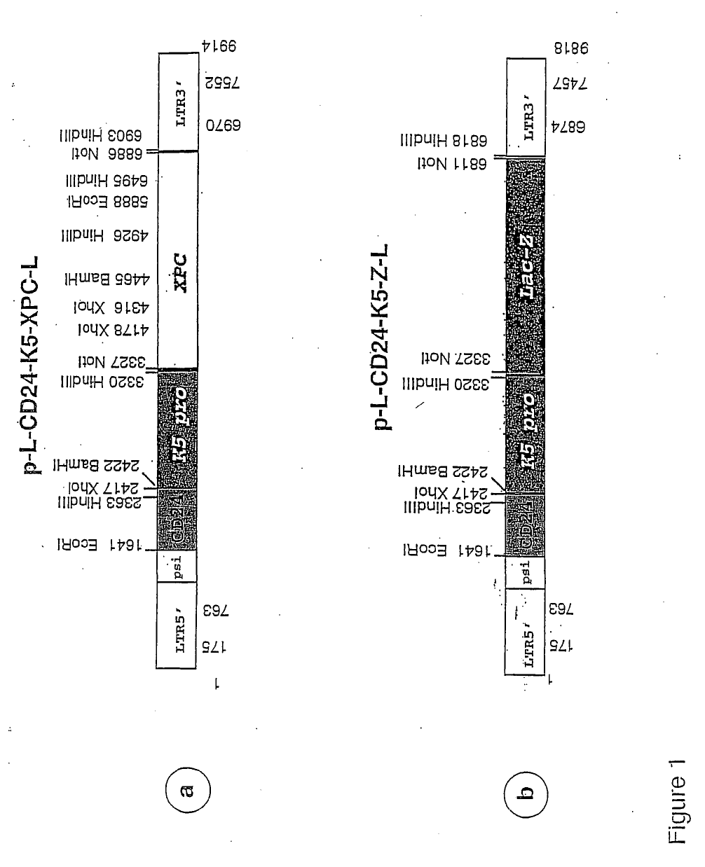

We have constructed by genetic engineering novel recombinant proviral DNA, derived from the proviral DNA of MoMLV (FIG. 1a, 1b). In these constructions, the CD24 is expressed from its complementary DNA the transcription of which is controlled upstream by the promoter present in the LTR 5′ of the proviral DNA of MoMLV. On the other hand, an internal promoter, the human keratin K5 promoter gene (K5 promoter), is placed upstream of the XPC complementation gene. The K5 promoter allows the targeting of the expression of the XPC gene in the basal cells, proliferative in the epidermis, since the latter are at the origin of cutaneous carcinomas (basal and spinal cell epitheliomas). The XPC gene is intended to complement the deficiency in the repair of damage induced by ultraviolet B radiation (290-320 nm) which is responsible for the photosensitivity and the predisposition to cutaneous carcinoma of keratinocytes in patients suffering from xeroderma pigmentosum (XP) belonging to the C complementation group. The XPC gene was chosen:

a-) because of the frequency of this complementation group in our region of the globe (>50%)

b-) because of the clinical presentation of the XP-C patients who are relatively free from other problems such as DNA repair deficiency, such as for example neurological degeneration. In fact, height-weight and neurological development is normal in these patients.

2. Applications Derived from the Construction

Using the “skeleton” of the construction shown in FIG. 1a, any other XP gene playing a role distinct from that of XPC in a DNA repair stage and being involved in XP pathology, can be inserted in place of XPC in order to ensure the complementation in the keratinocytes originating from patients belonging to the other XP complementation groups (i.e. XP-A, B, D, E, F, G).

Similarly, any other gene of interest (tracer gene, under the control of a promoter of interest, for example the bacterial lac Z gene), can be inserted in place of the XPC gene as illustrated in FIG. 1b.

Any other gene of interest, wild-type or carrying a mutation inactivating its original function, can also be inserted in place of the XPC gene illustrated in FIG. 1a, in order to study the effects, either of the overexpression of this gene, or of a given genetic mutation, determined for experimental purposes (analysis of the structure function relationships of the gene), or identified from human (tumor) samples. This latter example of use allows the production of mutant cells and the construction, from a single strain of wild-type keratinocytes of different strains of keratinocytes from the same genetic base but having different mutations on the gene of interest. If the mutations relate to a DNA repair gene, the effect of these mutations on the capacity of the cells to repair UV-induced lesions in the DNA can for example be measured and, if appropriate, the predisposition to cutaneous carcinoma of the keratinocytes carrying the mutation can be prognosticated.

Any other promoter of interest, can on the other hand be inserted in place of the keratin K5 promoter gene in order to control upstream the transcription of any gene of interest: complementation gene, tracer gene, reporter gene, or mutated gene.

3. Process for Transformation of the Keratinocytes and for Selection of the Transformed Stem Keratinocytes

The freshly inoculated keratinocytes (24 hours before infection) are incubated for 16-18 hours in the presence of infectious supernatant (containing the viral particles and 5 μg/ml of polybrene, hexadimetrine bromide). The infectious supernatant was obtained beforehand by overnight incubation of the cells which produce the viral particles in “SFM-medium”, (GIBCO Bethesda Research Laboratories, USA), 0.1 mM CaCl2, with no other nutrients.

After infection the keratinocytes are dissociated by enzymatic treatment with trypsin, resuspended at the rate of 450,000 cells/ml of culture medium of the keratinocytes containing 10% of decomplemented foetal calf serum (CFAD-DBS). The cells are incubated at 4° C. for 1.5 hours in the presence of 22 μg/10⁶ cells of anti-CD24 primary antibodies (clone ALB9, Immunotech, Luminy France). The cells are then washed twice for 10 minutes in CFAD-DBS then incubated in the presence of an excess (×44) of secondary antibodies coupled to metal beads (810 ⁷, M-450, DYNAL) previously washed in phosphate buffer (PBS) containing 0.1% of bovine serum albumin (BSA). The cells complexed with the antibodies coupled to the metal beads are separated from the other cells using a magnet. This stage is repeated twice. The complexed cells thus separated constitute the CD24-positive fraction. The non-complex cells constitute the CD24-negative fraction.

The CD24-positive cells are then dissociated from the antibodies/metal beads complex by an incubation for 45 minutes in the presence of a third “dissociation” antibody (Detachbead CD19, M-450, Pan-B DYNAL).The cells thus dissociated are then separated from the antibodies/metal beads complex, counted then inoculated for subsequent analysis: measurement of the expression of the gene of interest, functionality of the product of the gene of interest, ability to reconstitute a culture epithelium and a three-dimensional epidermis.

Certain cell-sorting processes, using cell sorters which allow the fixing of the positive cells' purity threshold up to a purity of >99.8%.

4. Processes for Preparation of Epidermis Reconstituted from Transformed Stem Keratinocytes

Several processes are applicable:

simple culture epithelium cultured from transduced cells and selected, inoculated and then taken to confluence. The culture epithelium is detached from the plastic support after treatment by the “dispose II” enzyme (12.5 mg/ml) and applied to the bed of the transplant. This standard technique is used for autografts in the treatment of large burns (Gallico et al., N. Engl. Med. 311, 448-451).

Another technique which can be used is that based on the production first described by Bell et al., 1979 Proc Natl Acad Sci USA 76, 274-1278. and improved by Asselineau et al., 1984, Exp. Cell. Res, 159, 536-539.

Diagram of the Novel Vectors

FIG. 1: Retroviral constructions. CD24: CD24 cDNA; XPC and lac Z: cDNAs of the genes of interest. K5 pro: 905 bp region promoting the transcription of the human keratin K5 gene. LTR 5′, LTR 3′: “long terminal region” of the proviral DNA of MoMLV. The LTR 5′ allows the transcription of the cDNA CD24. The recombinant proviral DNAs are derived from the vector LXSN (Clontech, accession #m28248).

1. Use of the CD24 marker gene of mammals, such as the human CD24 gene, for the transformation of stem keratinocytes in the implementation of processes for preparing homogenous batches of epidermal stem cells transformed in order to contain an exogenous nucleotide sequence of interest.

2. Process for preparing homogenous batches of epidermal stem cells transformed in order to contain an exogenous nucleotide sequence of interest, characterized in that it comprises the following stages:
the transformation of cells of the epidermis in culture comprising stem keratinocytes and post-mitotic keratinocytes of the suprabasal layer of the epidermis naturally secreting the CD24 protein using an appropriate vector the genome of which comprises a recombinant sequence containing an exogenous nucleotide sequence of interest and the CD24 gene as selection marker, which leads to the obtaining of a heterogeneous batch of cells, comprising the above-mentioned stem and post-mitotic keratinocytes transformed or not transformed with the recombinant sequence of the above-mentioned vector,
selection of the cells transformed during the preceding stage and expressing at their surface the CD24 protein, using a reagent specifically recognizing this protein, such as CD24-specific antibodies, which leads to the obtaining of a heterogeneous batch of cells comprising above-mentioned stem and post-mitotic keratinocytes transformed with the recombinant sequence of the above-mentioned vector, and if appropriate, of above-mentioned post-mitotic keratinocytes not transformed with said recombinant sequence,
the culture of the cells selected during the preceding stage for a period of time sufficient to allow the elimination of the above-mentioned post-mitiotic keratinocytes, transformed or not transformed with said recombinant sequence, capable of being present during the preceding stage of transformation and being incapable of dividing and therefore of being maintained in the culture medium, which leads to the obtaining of a homogenous batch of CD24⁺ stem keratinocytes containing said recombinant sequence, capable of dividing and of being maintained in culture.

3. Process according to claim 2, characterized in that the exogenous nucleotide sequence of interest is chosen from:
the nucleotide sequences capable of being used in gene therapy,
the nucleotide sequences which can be used as tracer genes to follow the development of a cell or population of transduced cells expressing this marker gene,
the nucleotide sequences comprising a dominant genetic mutation with phenotype effect, the effects of which on the physiology of the tissue can be studied,
the nucleotide sequences which can allow the screening of molecules of therapeutic interest for the epidermis, or of cosmetological interest.

4. Process according to claim 2, characterized in that the stage of selection of the cells transformed using CD24-specific antibodies is carried out using antibodies specifically recognizing the CD24 antibodies and coupled to magnetic beads, the CD24+ marked cells being separated from the non-marked cells using a magnet.

5. Stem keratinocytes from mammals, and more particularly humans, transformed in order that their genome contains the CD24 marker gene of mammals, and more particularly the human CD24 gene.

6. Stem keratinocytes transformed according to claim 5, characterized in that their genome contains at least one exogenous nucleotide sequence of interest as defined in claim 3.

7. Homogenous batch of stem keratinocytes transformed according to claim 5, the homogeneity rate of which is advantageously above approximately 99%, namely the proportion of non-transformed stem keratinocytes and/or of transformed or non-transformed post-mitotic keratinocytes is nil or lower than approximately 1%.

8. Transformed stem keratinocytes and homogenous batches of the latter, as obtained by implementation of a process according to claim 2.

9. Epidermal tissue reconstituted from transformed stem keratinocytes or homogenous batches of the latter according to claim 5.

10. Graft of epidermal tissue reconstituted according to claim 9.

11. Use of epidermal tissue reconstituted according to claim 9, for the preparation of grafts according to claim 10, intended for the treatment of pathologies linked to a genetic disorder.

12. Use of transformed stem keratinocytes or homogenous batches of the latter according to claim 5, for the implementation of:
processes for monitoring the in vitro development of a cell or population of transduced cells expressing exogenous nucleotide sequences which are of interest as marker genes,
processes for studying in vitro the effects on the physiology of the epidermal tissue of exogenous nucleotide sequences of interest corresponding to sequences comprising a dominant genetic mutation with phenotype effect,
processes for screening in vitro molecules of therapeutic interest for the epidermis, or of cosmetological interest, capable of having an effect directly on the exogenous nucleotide sequence of interest, or indirectly on the cell physiological state induced by said exogenous nucleotide sequence of interest, the latter processes comprising a stage of placing said molecules of interest in the presence of the transformed stem keratinocytes or the above-mentioned transformed epidermal tissue.

13. Use of epidermal tissue reconstituted according to claim 9, for the implementation of:
processes for monitoring the in vitro development of a cell or population of transduced cells expressing exogenous nucleotide sequences which are of interest as marker genes,
processes for studying in vitro the effects on the physiology of the epidermal tissue of exogenous nucleotide sequences of interest corresponding to sequences comprising a dominant genetic mutation with phenotype effect,
processes for screening in vitro molecules of therapeutic interest for the epidermis, or of cosmetological interest, capable of having an effect directly on the exogenous nucleotide sequence of interest, or indirectly on the cell physiological state induced by said exogenous nucleotide sequence of interest, the latter processes comprising a stage of placing said molecules of interest in the presence of the transformed stem keratinocytes or the above-mentioned transformed epidermal tissue.
